(19) United States
(12) Patent Application Publication
(10) Pub. No.: US 2020/0330179 A1
(43) Pub. Date: Oct. 22, 2020

(54) SURGICAL LOUPES USING A PRISM AND ADDITIONAL FEATURES

(71) Applicant: Trieu TON, Brentwood, CA (US)

(72) Inventor: Trieu TON, Brentwood, CA (US)

(21) Appl. No.: 16/663,643

(22) Filed: Oct. 25, 2019

Related U.S. Application Data
(60) Provisional application No. 62/836,140, filed on Apr. 19, 2019.

(51) Int. Cl. A61B 90/00; G02C 7/08; G02B 25/00; G02B 25/02

(52) U.S. Cl. CPC A61B 90/361; G02C 7/088; G02B 25/004; A61B 2090/502; A61B 2090/3616; A61B 2090/3618; G02B 25/02

(57) ABSTRACT

A pair of surgical loupes adapted to be secured to an eyewear portion. An embodiment of the surgical loupes comprises mirror coated Littrow lenses and double concave or triplet lenses. Another embodiment of the surgical loupes and eyewear portion comprises a vibration mechanism operably connected to the eyewear portion. Another embodiment of the surgical loupes comprises a counterweight mechanism operably connected to the eyewear portion. Another embodiment of the surgical loupes comprises a LED and camera portion. The LED light may be an orange spectrum light that blocks out blue spectrum light so as to prevent premature curing during dental procedures. Still yet another embodiment of the surgical loupes comprises blue spectrum light blocking lenses operably location in the lens housing.

This application claims the benefit of U.S. Provisional Patent Application Ser. No. 62/836,140 filed Apr. 19, 2019, the contents of which are hereby incorporated herein by reference.

### BACKGROUND

### 1. Field

This application is directed to the field of surgical gear. In particular, the application is directed to surgical loupes and eyewear used in the field of surgery.

### 2. Description of the Related Art

Surgeons, dentists and hygienists use surgical loupes to magnify the area where they perform theft work. Traditional surgical loupes use a telecentric design where the user has to tilt their head downward so as to view the working area below them. The tilting of their head can lead to neck and back pain. For people in these professions, according to the American Dental Association, roughly 29% of disabilities are related to musculoskeletal disorders. These disabilities can lead to early retirement and loss of income.

### SUMMARY

Briefly described, aspects of the present disclosure relate to surgical loupes and eyewear.

An aspect of the present disclosure is surgical loupes. The surgical loupes have a housing portion, wherein the housing portion comprises a bridge, wherein a first lens housing is located at a distal end of the bridge and a second lens housing is located at an opposite distal end of the bridge, wherein the housing portion further comprises an attachment member adapted to be secured to eyewear; wherein each of the first lens housing and the second lens housing have located therein a mirror coated Littrow prism, wherein the mirror coated Littrow prism reflects light from the mirror coated portion of the Littrow portion through the hypotenuse portion the Littrow prism to eyes of a user; and wherein each of the first lens housing and the second lens housing have located therein at least one plano convex lens.

Another aspect of the present disclosure is a system. The system has an eyewear portion, surgical loupes operably connected to the eyewear portion, wherein the surgical loupes comprise; a housing portion, wherein the housing portion comprises a bridge, wherein a first lens housing is located at a distal end of the bridge and a second lens housing is located at an opposite distal end of the bridge, wherein the housing portion further comprises an attachment member adapted to be secured to eyewear; wherein each of the first lens housing and the second lens housing have located therein a mirror coated Littrow prism, wherein the mirror coated Littrow prism reflects light from the mirror coated portion of the Littrow portion through the hypotenuse portion the Littrow prism to eyes of a user; and wherein each of the first lens housing and the second lens housing have located therein at least one double concave lens or triplet lens.

### BRIEF DESCRIPTION OF THE DRAWINGS

FIG. 1 is a top down view of surgical loupes.

FIG. 2 is a rear view of the surgical loupes shown in FIG. 1.

FIG. 3 is a front view of the surgical loupes shown in FIG. 1.

FIG. 4 is a side view of the surgical loupes shown in FIG. 1.

FIG. 5 is an internal view of an arrangement of lenses and a mirrored Littrow prism used with the surgical loupes.

FIG. 6 is an internal view of an arrangement of lenses and mirrored Littrow prism used with the surgical loupes that includes a lens for zooming in and out.

FIG. 7 shows a diagram of a Littrow prism.

FIG. 8 shows eyewear and surgical loupes.

FIG. 9 shows the eyewear and surgical loupes in a state where the loupes are not in position to look through.

FIG. 10 shows the eyewear and surgical loupes in a state wherein the loupes are ready for use.

FIG. 11 shows a screw mounted camera for use with the surgical loupes.

### DETAILED DESCRIPTION

To facilitate an understanding of embodiments, principles, and features of the present disclosure, they are disclosed hereinafter with reference to implementation in illustrative embodiments. Embodiments of the present disclosure, however, are not limited to use in the described systems or methods and may be utilized in other systems and methods as will be understood by those skilled in the art familiar with this disclosure.

Aspects of the present invention apply principles related to surgical loupes disclosed in Applicant's U.S. Pat. No. 10,247,965, incorporated herein by reference. The surgical loupe design utilizes a lens arrangement and an aluminum mirror coated Littrow prism to redirect a user's view and magnify the area where the user is working. This is accomplished while still allowing the user to maintain a straight neck and head posture.

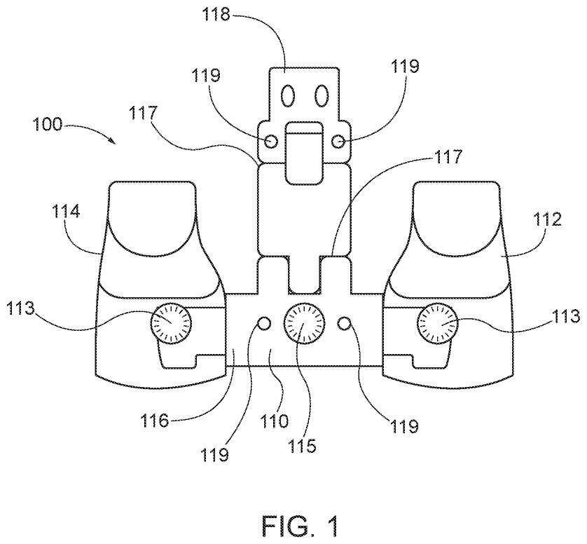

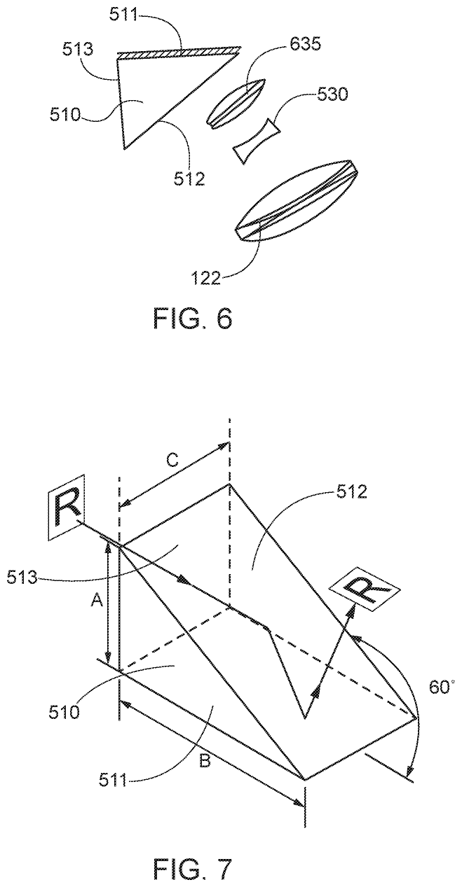

Referring to FIGS. 1.4, in an embodiment, the surgical loupes 100 of the disclosure use a mirror coated Littrow prism (shown in FIGS. 5 and 6) coupled with a double concave lens or triplet lens to redirect the user's view and magnify the working area, while allowing the user to maintain a straight neck and head posture. The surgical loupes may use triplet lens which comprises a double concave lens and a plano convex lens. In an embodiment, there is a 2.5 degrees tilt built into the housing for the lens arrangement allowing the images to converge. In an embodiment, the surgical loupes have the ability to achieve multiple magnification states.

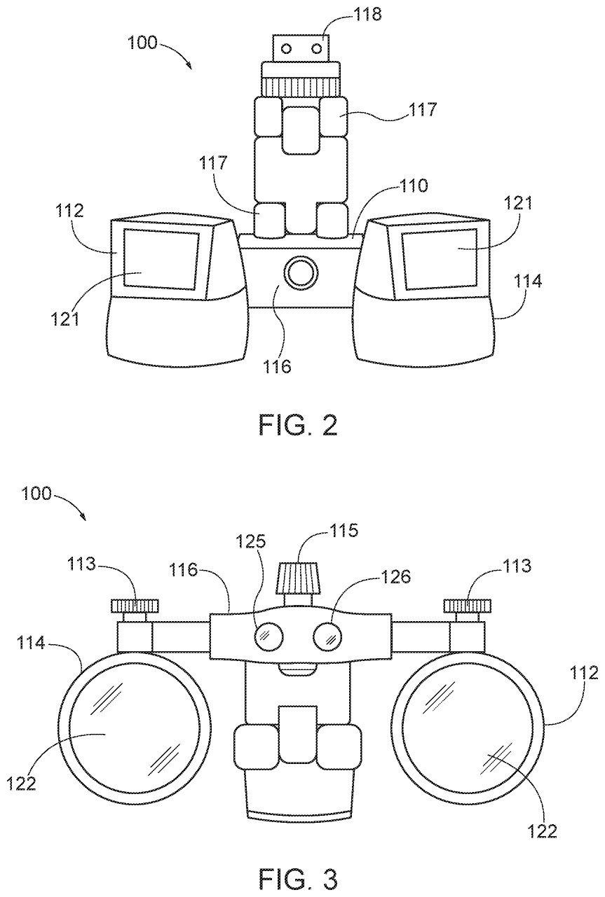

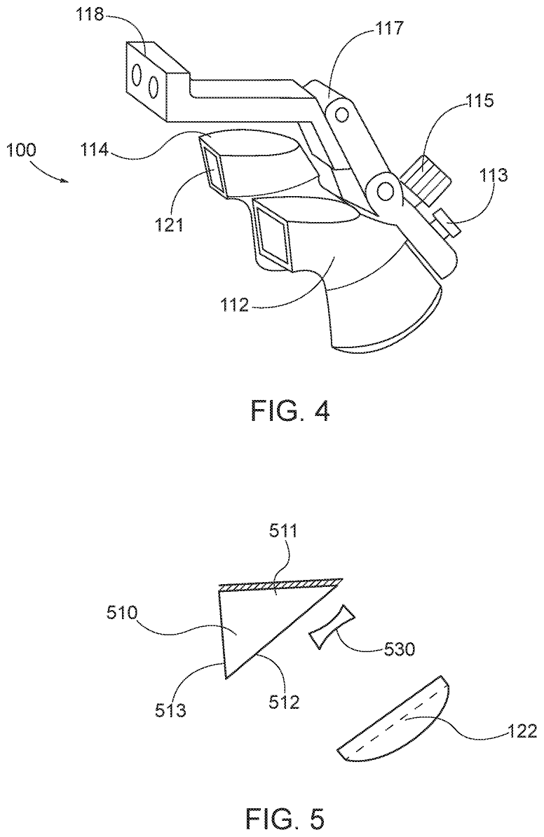

Still referring to FIGS. 1-4, the surgical loupes 100 have a housing 110. In an embodiment, the housing 110 is made of plastic material. In an embodiment, the housing 110 is made of metal material. In an embodiment, the housing 110 is made of ceramic material. In an embodiment, the housing 110 is made of a composite of more than one material.

Operably connected to the housing 110 is a left lens housing 112 and right lens house 114. The left lens housing 112 and right lens housing 114 are operably connected to the housing 110 via adjustable screws 113. The adjustable screws 113 can be used to adjust the orientation of the left lens housing 112 and right lens housing 114 with respect to the housing 110. Movement of the adjustable screws 113 permit the respective right lens housing 114 and left lens housing 112 to move so that better viewing can be achieved. After adjustment, the left lens housing 112 and the right lens housing 114 are secured in place. The left lens housing 112 and the right lens housing 114 each have an intake lens 122 through which light passes through. The left lens housing 112 and the right lens housing 114 each have a viewing lens 121 though which light exits and the user looks when using the surgical loupes 100.

In an embodiment, instead of an adjustable screw there is a sliding mechanism. In an embodiment, instead of an adjustable screw there is an adjustable dial. In an embodiment, instead of an adjustable screw there is a button that activates movement of lens within the left lens housing and the right lens housing. In an embodiment, the focusing and adjustment of lens housings are activated by voice commands that are picked up by a microphone on the surgical loupes. In an embodiment, the adjustment of lens housings is activated by voice commands that are picked up by a smartphone that is connected via Bluetooth to the surgical loupes, which has a processor that processes voice commands and uses the voice commands to adjust the lens housings.

Located on the top portion of the housing 110 is an adjustable dial 115 that is located on the bridge 116 of the housing 110. The adjustable dial 115 is able to adjust the focusing and magnification ability of the surgical loupes 100. The housing 110 further comprises hinged portions 117 that permit the bridge 116 to be moved and adjusted. The housing 110 further has an attachment member 118 that permits the surgical loupes 100 to be attached to eye gear, such as glasses. In an embodiment. different magnifications of 2×, 2.5×, 3.5×, 4× and gradual zoom are available. In an embodiment. different magnifications of 2× to 10× and gradual zoom are available. In an embodiment, gradual zoom of 2×-6× is used. In an embodiment, gradual zoom of 3×-10× is used. Having multiple magnification settings in one set of surgical loupes eliminates the need to purchase multiple sets of loupes.

In an embodiment, instead of an adjustable dial there is a sliding mechanism. In an embodiment, instead of an adjustable dial there is an adjustable screw. In an embodiment, instead of an adjustable dial there is a button that activates focusing of the lenses. In an embodiment, the focusing and magnification with the lenses is activated by voice commands that are picked up by a microphone on the surgical loupes.. In an embodiment, the focusing and magnification with the lenses are activated by voice commands that are picked up by a smartphone that is connected via Bluetooth to the surgical loupes, which has a processor that processes voice commands and uses the voice commands to adjust focus of the lenses.

Additionally, located on the bridge 116 may be magnets 119 that are used to secure portions of the surgical loupes 100 to other portions of the surgical loupes 100. For example, the magnets 119 located on the bridge may be secured to magnets 119 located on the attachment member 118. In an embodiment, the magnets may be electromagnets that are activated by a switch or button. In an embodiment, the magnets are used to secure the lens housings in place. In an embodiment, the magnets are located on the lens housings. In an embodiment, the magnets are located on the top of the bridge.

In an embodiment, the magnets are located on the bottom of the bridge. In an embodiment, the magnets are located on the hinged portions of the housing. In an embodiment, the magnets are located on each portion of the housing further used to secure portions of the surgical loupes to portions of the eyewear. It should be understood that any number of magnets may be used for securing the surgical loupes.

Still referring to FIGS. 1-4, in an embodiment, the surgical loupes 100 implement a light 125. In an embodiment, the light 125 is a light emitting diode (LED) light. The color rendering index (CRI) of the LED depends on the LED, battery source power. Having a high CRI (greater than 90) is preferable. In an embodiment, the light 125 is a bulb. In an embodiment, the light 125 is an optical fiber that is adapted to be movable with respect to the housing 110. Located within the housing 110 is a power source (not shown) that is operably connected to the light 125 so as to power the light 125. In an embodiment, the power source is a chemical power source. In an embodiment, the power source is solar power source. In an embodiment, the power source is a lithium-based power source. In an embodiment, the power source is a kinetic based power source. The light 125 is adapted to illuminate the working space where the user of the surgical loupes is working.

In an embodiment, the light 125 has a built-in orange light filter to permit dentists (or other surgical personnel) to illuminate their working area. The use of a built-in orange light filter helps avoid curing composite resins or cements that can be cured by certain types of lights, such as blue spectrum lights. An orange light filter will absorb light in the blue spectrum, thereby preventing blue spectrum light from reaching the working area. In an embodiment, the light 125 can emit light or filter light in other color spectrums. in an embodiment, the light 125 emits ultra-violet light. In an embodiment, voice commands are used to adjust the brightness of the lights, In an embodiment, a dial or sliding switch is used to adjust the brightness of the light In an embodiment, the light can be switched to different optical spectrums. In an embodiment, the light is adjusted from low, medium, high, and ultra-bright. In an embodiment, the light is concentrated spotlight to focus brightness in a certain area.

In an embodiment, the surgical loupes implement a camera 126. In an embodiment, the surgical loupes 100 implement a light 125 in addition to a camera. In an embodiment, the camera 126 is embedded in the housing 110 of the surgical loupes 100 and permits a user to record their surgery. In an embodiment, the camera 126 is removably attached to the surgical loupes 100. In an embodiment, the camera 126 is wirelessly adapted to transmit signals to a computer to record a surgery. In an embodiment, the camera 126 has Bluetooth capability and is able to connect to various devices in the area. In an embodiment, the camera 126 has magnification capabilities. In an embodiment, wires for the light 125 and the camera are located within the housing 110 to provide a dean, modern look. In an embodiment, the light 125 or the camera 126 or both can be activated manually by touch or voice command. When operated by voice command a microphone is implemented and operably located on the housing 110 or the left lens housing 112 or the right lens housing 114.

Referring to FIGS. 5 and 6, located within the left lens housing 112 or the right lens housing 114, shown in FIGS. 1-4, is a Littrow prism 510 that has one side mirrored. In an embodiment, the Littrow prism 510 is 30-60-90 degree Littrow prism. In FIGS. 5 and 6, the mirror coated side 511 is the side of the Littrow prism 510 that is opposite the hypotenuse side 512 and the 60° angle of the Littrow prism 510. In an embodiment, light enters the Littrow prism 510 through the hypotenuse side 512 strikes the mirror coated side 511 and exits through another portion of the hypotenuse side 512. In an embodiment, light enters the Littrow prism 510 through the hypotenuse side 512 strikes the mirror coated side 511 and exits through the third side 513 that is opposite the 30° degree angle. In an embodiment, the mirrored side is a side different than the side opposite the 60° angle of the Littrow prism. In an embodiment, the mirrored side is a side opposite the 30° angle of the Littrow prism. In an embodiment, the mirrored side is a side opposite the 90° angle of the Littrow prism. In an embodiment, the prism is not a 30-60-90 degree prism. In an embodiment, the prism is a 20-70-90 degree prism. In an embodiment, the prism is a 10-80-90 degree prism. In an embodiment, the prism is a 45-45-90 degree prism. In an embodiment, the prism is 35-55-90 degree prism. In an embodiment, the prism is 25-65-90 degree prism. In an embodiment, the prism is 15-75-90 degree prism. In an embodiment, the prism is any prism where one of the angles is 90 degrees and the complementing angle sum to 90 degrees.

In an embodiment, the mirrored side is mirrored by being silver coated. In an embodiment, the mirrored side is mirrored by being aluminium coated. In an embodiment, the mirrored side is mirrored by being gold coated. In an embodiment, the mirrored side is mirrored by being UV-enhanced aluminium coated. In an embodiment the mirror coating is enhanced aluminium. In an embodiment, the mirror coating is delta UV enhanced aluminium. In an embodiment, the mirrored coating is bare gold. In an embodiment the mirror coating is protected gold. In an embodiment the mirror coating is protected silver. Protected aluminum and enhanced aluminum are typically used for visible applications. UV and DUV enhanced aluminium can be used for UV and visible applications. Bare or protected gold offers high reflectance for near—Infrared (NIR) and Infrared wavelengths. Protected silver provides the highest reflectance between 500-800 nm and also performs well in near-Infrared and Infrared applications.

FIGS. 5 and 6 shows a double concave lens 530 used with the Littrow prism 510. The double concave lens 530 is adapted to move with respect to the Littrow prism 510 and allows the user to magnify the image. FIG. 6 shows an additional lens 635 that adapted to modify the view of the user so that it can be rotated, arranged and/or unified. In an embodiment, the additional lens 635 is a double lens. In an embodiment, the additional lens 635 is a triplet lens. In an embodiment, the additional lens is tilted double with a tilted double lens or triplet lens.

A doublet lens is a type of lens made up of two simple lenses paired together. Such an arrangement allows for more optical surfaces, thicknesses, and formulations, especially as the space between lenses may be considered an “element” with respect to the lens design. A triplet lens is a compound lens consisting of three single lenses. A triplet lens provides a number of degrees of freedom to allow the lens design to overcome potential Seidel aberrations. Overcoming potential aberrations sharpens the image along with displaying a larger viewing area.

Referring to FIG. 7, in operation, incident light that passes through the hypotenuse side 512 and strikes the mirrored coated surface 511 surface of the Littrow prism at a nominal angle, returns back using the same path. In spectrum dispersion applications utilizing white light, the resolution performance of Littrow prisms is equal to equilateral prisms since the optical path length through the glass substrate is the same distance round-trip. Additionally, light entering through the third side 513 will reflect twice inside the prism before being emitted through the hypotenuse side at 60°. The Littrow prism 510 will have a ray deviation of 60° when a mirror coated surface is used.

Referring generally to FIGS. 1-6, The surgical loupes 100, have a surface of the Littrow prism 510 coated with material so as to be mirror in a manner that will cause light impacting the mirror coating to be reflected towards the interior of the Littrow prism 510. The mirror coating allows reflections and prevents having ghost images. With first surface mirror coatings, the coated surface faces the incident light in order to minimize energy loss by not allowing light to pass through the glass substrate.

The mirror coated Littrow prism 510 is positioned proximate to a user's eye, with its slanted surface (the hypotenuse of the triangle formed by the cross-section of the prism) positioned proximate to viewing lens 121 through which the user looks. In an embodiment, an additional doublet lens is placed between the Littrow prism 510 and the viewing lens 121. In an embodiment, a triplet lens is placed between the Littrow prism 510 and the viewing lens 121. In an embodiment, both doublet and triplet lenses are used in various combinations between the Littrow prism 510 and the viewing lens 121. The lenses used depend on the desired magnification. In an embodiment, the lenses are positioned downward at an angle above one another. In an embodiment, the lenses are positioned centric to another with a tilt of approximately 60 degrees due to the ray deviation of the prism. In an embodiment, the lenses are grouped in three sections, which permits the user to rotate and shift the group of lenses inward or outward. The center lens or group of lenses can move as the left lens housing 112 or the right lens housing 114 is rotated. Groups of lenses permit reduction of aberrations and display a sharper image. Moving the respective location of the lenses permit the lenses to zoom in and out of the working area.

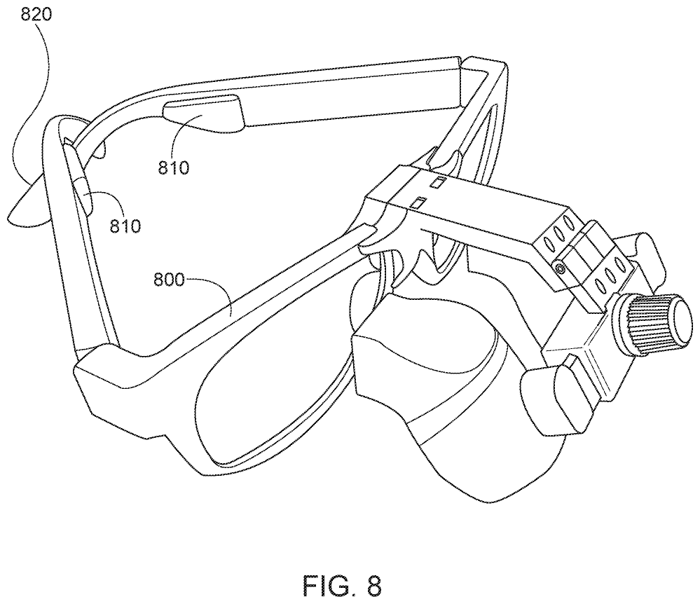

Referring to FIG. 8 for reference, in an embodiment, eyewear 800 is operably connected to a portion of the surgical loupes. In an embodiment, eyewear 800 has operably connected to it a vibration mechanism 810. The vibration mechanism 810 is adapted to transmit vibrations to portions of the ear that are able to translate the vibration into audio that is perceptible by the user. In an embodiment the vibrations are transmitted to bones proximate to the ear. In an embodiment, the vibrations are transmitted to ossicles. In an embodiment, the vibration mechanism 810 is operably attached to the eyewear portion 800. In an embodiment, the vibration mechanism 810 is removably attached to the eyewear portion 800. In an embodiment, the vibration mechanism is connected to the surgical loupes and the surgical loupes transmit signals to the vibration mechanism. In an embodiment, the eyewear portion is a pair of protective goggles. In an embodiment, the eyewear portion is a strap that is adapted to hold the surgical loupes. in an embodiment, the eyewear portion is a strap that has threaded therein a vibration mechanism adapted to fit next to a user's ears. In an embodiment the vibration mechanism transmits the vibrations directly to the ossicles. In an embodiment, the vibration mechanism is mechanically vibrated. In an embodiment, the vibration mechanism is electrically vibrated. In an embodiment, the vibration mechanism is a piezoelectric crystal that is electrically actuated.

Bone conduction audio allows users implementing the surgical loupes to transmit music and other sounds to the user's inner ear through vibration of the cochlea, without involving the eardrum. This allows the user of the surgical loupes implementing the vibration mechanism to be able to interact and be aware with theft surroundings, yet be able to listen to podcasts/music/audiobooks while doing surgery.

Still referring to FIG. 8, in an embodiment, to avoid having the surgical loupes and eyewear 800 slipping due to the weight, a counterweight 820 is operably attached to the eyewear 800. In an embodiment, the counterweight 820 is a soft rubber attachment that is adjustable, replaceable and operably connected to the eyewear. In an embodiment, the counterweight is slidable onto both sides of the back of the eyewear portion 800. When the user wears them, the attachments will be nested onto the back of the user's ears. This prevents slippage and a comfortable, stable fit for the user. This avoids problems that previously existed in the past where users would have to tighten eyewear straps to secure their loupes and eyewear to theft head. Over time, these straps would get loose and separate from the eyewear. The counterweight prevents the surgical loupes and eyewear from falling forward. in an embodiment, the counterweights are adapted to counterbalance the weight of the loupes. In an embodiment, there is a silicone earpiece hinge that can be hooked onto the ears. In an embodiment, the silicone earpiece hinge is used in conjunction with a strap or other mechanism to secure the eyewear portion to the head during use.

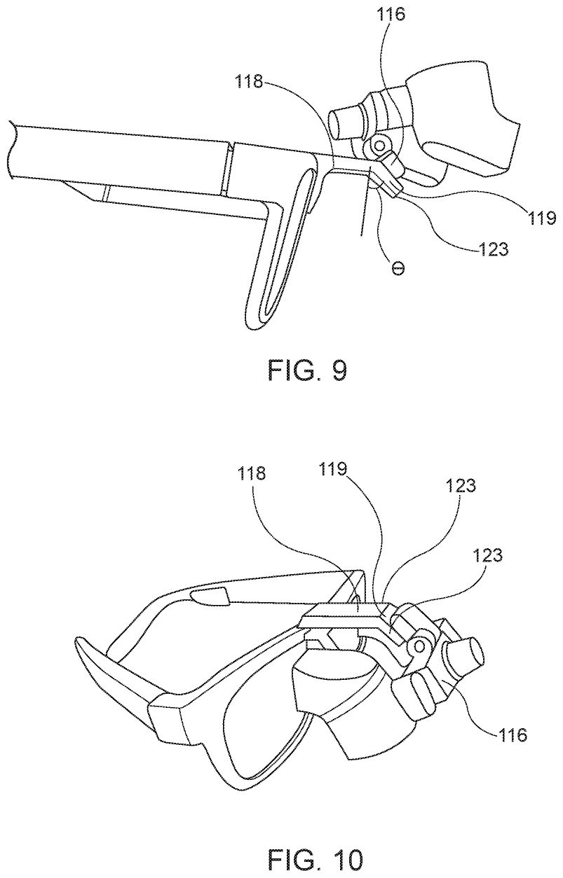

Turning to FIGS. 9 and 10, an example of the surgical loupes 100 employing magnets 119 is shown. in FIG. 9 the surgical loupes 100 are shown in a raised position where the magnets located on the attachment member 118 are secured to the magnets located on the bridge 116. FIG. 10 shows the surgical loupes 100 in a lowered position, which is the position where the surgical loupes 100 are able to be used by a user for surgical procedures, etc. FIGS. 9 and 10 further shows an angled portion 123 of attachment member 118. The angled portion 123 is angled with respect to the horizontal portion of the attachment member 118 so that the angled portion 123 extends in a downward direction when the surgical loupes 100 are worn. The angled portion 123 extends downwardly from the horizontal portion of the attachment member 118 at an angle θ that is at 45° with respect to a perpendicular line that extends perpendicularly from the horizontal portion of the attachment member 118. By having the angled portion 123 extend at an angle the surgical loupes 100 are better secured by the magnets 119 so that they do not fall downward when they are to be secured in the raised position. This is do to taking advantage of gravity in assisting the securing of the surgical loupes 100. In an embodiment, the angled portion 123 extends downwardly from the horizontal portion of the attachment member 118 at an angle θ that is at 35° with respect to a perpendicular line that extends perpendicularly from the horizontal portion of the attachment member 118. In an embodiment, The angled portion 123 extends downwardly from the horizontal portion of the attachment member 118 at an angle θ that is at 25° with respect to a perpendicular line that extends perpendicularly from the horizontal portion of the attachment member 118. The angled portion 123 extends downwardly from the horizontal portion of the attachment member 118 at an angle θ that is at least 15° with respect to a perpendicular line that extends perpendicularly from the horizontal portion of the attachment member 118. In an embodiment, the angled portion 123 extends downwardly from the horizontal portion of the attachment member 118 at an angle θ that is between 5° and 85° with respect to a perpendicular line that extends perpendicularly from the horizontal portion of the attachment member 118.

In an embodiment, lenses may be inserted within the arrangement of the lenses for the surgical loupes that block blue spectrum light. Blue spectrum light blocking lenses are inserted into the surgical loupes to reduce harmful blue lights. Constant viewing of blue light from digital devices, sunlight, mobile devices, computer monitors, and LED illumination light at the surgery site can cause eye stains/damage and disrupt sleep patterns and the circadian cycle. In dentistry blocking blue spectrum light further prevents viewing curing lights that are within blue light emitting devices used to activate and cure composite filling materials and cement. Looking at them with regular clear glasses can damage a user's eyes.

In an embodiment, the anti-blue spectrum light protection is coated along with UV protection, to protect against light in the ultraviolet and blue-violet spectrum can damage the human eye, as well as leading to painful inflammation of the conjunctiva and cornea. The coatings can be applied on the loupes lenses so that the user doesn't magnify the entrance of the blue light. Similar to holding a magnifying glass and converging the rays into a point, which can generate a lot of heat.

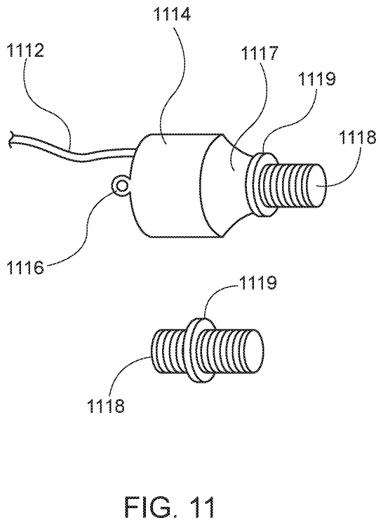

Referring now to FIG. 11, shown is a camera 1114 that is mountable to the surgical loupes. The camera 1114 has a cable 1112 that is operably connected to the surgical loupes. In an embodiment, the cable 1112 is not present and the camera 1114 is battery operated and uses wireless communication in order to transmit recorded information and run the camera. In an embodiment, the camera is able to be connected directly to a mobile phone. In an embodiment, the camera is able to wirelessly connect to a mobile phone. In an embodiment, the camera is able to connect to a mobile phone via micro-usb. In an embodiment, the camera is able to connect to a mobile phone via usb. In an embodiment, the camera is able to connect to a mobile phone via an iPhone connector. In an embodiment, the camera is able to connect to a computer via the same manner in which it connects to the mobile phone. In an embodiment, the camera is able to connect to a mobile phone or computer using Bluetooth or some other wireless protocol.

Mounting portion 1116 permits the camera 1114 to be secured to a portion of the surgical loupes. Adjustment screw 1117 permits the lens portion 1118 to be adjusted via adjustment screw 1117. By twisting the adjustment dial 1119 the lens portion 1118 moves with respect to the adjustment screw 1117. The lens portion 1118 is able to be moved back and forth with respect to the area that is being recorded thereby adjusting the depth and focus of the camera 1114. Additionally, the lens portion 1118 is able to be removed and lenses with different zoom capabilities are able to be obtained. For example a 2× or 3× lens may be used. In some embodiments, lenses within the range of 1.5× to 10× are able to be used with the camera 1114. The camera 1114 is adapted to be used with any of the surgical loupes discussed above.

While embodiments of the present disclosure have been disclosed in exemplary forms, it will be apparent to those skilled in the art that many modifications, additions, and deletions can be made therein without departing from the spirit and scope of the invention and its equivalents, as set forth in the following claims.

What is claimed is:

1. Surgical loupes comprising:
a housing portion, wherein the housing portion comprises a bridge, wherein a first lens housing is located at a distal end of the bridge and a second lens housing is located at an opposite distal end of the bridge, wherein the housing portion further comprises an attachment member adapted to be secured to eyewear;
wherein each of the first lens housing and the second lens housing have located therein a mirror coated Littrow prism, wherein the mirror coated Littrow prism reflects light from the mirror coated portion of the Littrow portion through the hypotenuse portion the Littrow prism to eyes of a user; and
wherein each of the first lens housing and the second lens housing have located therein at least one plano convex lens.

2. The surgical loupes of claim 1, wherein the surgical loupes are adapted to change magnification.

3. The surgical loupes of claim 1, further comprising a camera operably located on the bridge.

4. The surgical loupes of claim 1, wherein each of the first lens housing and the second lens housing have located therein lenses that block blue spectrum light.

5. The surgical loupes of claim 1, wherein the bridge has an LED light operably connected thereto.

6. The surgical loupes of claim 5, wherein the LED light emits a light in the orange spectrum.

7. The surgical loups of claim 6, wherein the orange spectrum light is adapted to prevent curing of dental resins.

8. The surgical loupes of claim 1, wherein the bridge has magnets located thereon.

9. The surgical loupes of claim 8, wherein the attachment member has magnets located thereon that are adapted to be magnetically secured to the magnets located on the bridge.

10. The surgical loupes of claim 1, further comprising a microphone operably connected to a processor adapted to activate a camera or LED light.

11. A system comprising:
an eyewear portion,
surgical loupes operably connected to the eyewear portion, wherein the surgical loupes comprise;
a housing portion, wherein the housing portion comprises a bridge, wherein a first lens housing is located at a distal end of the bridge and a second lens housing is located at an opposite distal end of the bridge, wherein the housing portion further comprises an attachment member adapted to be secured to eyewear;
wherein each of the first lens housing and the second lens housing have located therein a mirror coated Littrow prism, wherein the mirror coated Littrow prism reflects light from the mirror coated portion of the Littrow portion through the hypotenuse portion the Littrow prism to eyes of a user; and
wherein each of the first lens housing and the second lens housing have located therein at least one double concave lens or triplet lens.

12. The system of claim 11, further comprising a vibration mechanism operably attached to the eyewear portion, wherein the vibration mechanism is adapted to transmit vibrations through the ossicles of a user's ears.

13. The system of claim 11, further comprising counterweights operably connected to the eyewear portion, wherein the counterweights are adapted to counter weight of the surgical loupes.

14. The system of claim 11, wherein the surgical loupes are adapted to change magnification.

15. The system of claim 11, further comprising a camera operably located on the bridge.

16. The system of claim 11, wherein each of the first lens housing and the second lens housing have located therein lenses that block blue spectrum light.

17. The system of claim 11, wherein the bridge has an LED light operably connected thereto.

18. The system of claim 17, wherein the LED light emits a light in the orange spectrum.

19. The system of claim 18, wherein the orange spectrum light is adapted to prevent curing of dental resins.

20. The system of claim 11, wherein the bridge has magnets located thereon.
